(19) United States
(12) Patent Application Publication
(10) Pub. No.: US 2026/0315270 A1
(43) Pub. Date: Oct. 15, 2026

(54) HIGH-PRESSURE MEDICAL CRYOTHERAPY DEVICE

(71) Applicant: Gregory Papageorge, Old Westbury, NY (US)

(72) Inventor: Gregory Papageorge, Old Westbury, NY (US)

(21) Appl. No.: 19/716,884

(22) Filed: Jun. 23, 2026

Related U.S. Application Data
(63) Continuation-in-part of application No. 18/605,136, filed on Mar. 14, 2024.
(60) Provisional application No. 63/452,584, filed on Mar. 16, 2023.

(51) Int. Cl. A61F 7/00; A61F 7/02

(52) U.S. Cl. CPC A61F 7/007; A61F 2007/0063; A61F 2007/0071; A61F 2007/0095; A61F 2007/0282

(57) ABSTRACT

A high-pressure medical cryotherapy device is provided. The device is designed for spraying liquid carbon dioxide onto a patient to alleviate inflammation through a series of treatments via a portable machine. The device features a gun-like body with a quick-connect fitting for liquid CO2 entry, a trigger-operated valve system for CO2 flow control, and a nozzle that can be attached in various ways and adjusted for different spray patterns. Additional components include a flow adjustment knob for regulating CO2 flow rate, a heating element to prevent frosting and ensure smooth CO2 expulsion, thermistors, and temperature sensors for temperature management, and lasers for visual distance referencing.

### CROSS-REFERENCE TO RELATED APPLICATION

The present application is a continuation-in-part patent application of U.S. patent application Ser. No. 18/605,136, filed on Mar. 14, 2024, which claims priority to, and the benefit of, U.S. Provisional Application No. 63/452,584, which was filed on Mar. 16, 2023, both of which incorporated herein by reference in their entirety.

### FIELD OF THE INVENTION

The present invention relates generally to the field of cryotherapy. More specifically, the present invention relates to a portable gun device designed for spraying liquid carbon dioxide onto a patient to alleviate inflammation through a series of cryotherapy treatments. Accordingly, the present disclosure makes specific reference thereto. Nonetheless, it is to be appreciated that aspects of the present invention are also equally applicable to other like applications, devices, and methods of manufacture.

### BACKGROUND

Cryotherapy has become a widely recognized and popular method for treating various types of body tissue, offering benefits that range from reducing inflammation to alleviating pain and speeding up recovery after exercise. The principle behind cryotherapy involves exposing the body to extremely cold temperatures for short periods, which can stimulate physiological responses that are therapeutic in nature. Despite its growing popularity and proven benefits, a significant limitation exists in the current landscape of cryotherapy technology.

The machines designed to administer cryotherapy treatments are typically extremely large and cumbersome. This substantial size not only makes them a significant investment in terms of space within a facility but also severely limits their portability. Consequently, this lack of portability presents a considerable drawback for users who require the flexibility of receiving cryotherapy treatments at various locations. Whether for athletes who travel frequently for competitions, individuals who split their time between multiple residences, or professionals who could benefit from the convenience of on-site treatments in different settings, the inability to easily transport cryotherapy machines restricts access to continuous and consistent care.

Furthermore, the impracticality of moving these large machines from one location to another means that users are often required to seek out specialized facilities that offer cryotherapy. This can result in additional challenges, such as scheduling appointments around availability and traveling to and from these facilities, which could deter individuals from pursuing cryotherapy as a regular part of their wellness or recovery routines.

Therefore, there exists a long-felt need in the art for an improved cryotherapy device. There also exists a long-felt need in the art for a high-pressure medical cryotherapy device that is portable. In addition, there exists a long-felt need in the art for a high-pressure medical cryotherapy device that is well-built and durable.

The subject matter disclosed and claimed herein, in one embodiment thereof, comprises a high-pressure medical cryotherapy device. The device is designed for spraying liquid carbon dioxide onto a patient to alleviate inflammation through a series of treatments via a portable machine. The device features a gun-like body with a quick-connect fitting for liquid CO2 entry, a trigger-operated valve system for CO2 flow control, and a nozzle that can be attached in various ways and adjusted for different spray patterns. Additional components include a flow adjustment knob for regulating CO2 flow rate, a heating element to prevent frosting and ensure smooth CO2 expulsion, thermistors, and temperature sensors for temperature management, and lasers for visual distance referencing.

In this manner, the high-pressure medical cryotherapy device of the present invention accomplishes all the forgoing objectives and provides an improved cryotherapy device. More specifically, the device provides a high-pressure medical cryotherapy device that is portable. In addition, the device is well-built and durable.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a high-pressure medical cryotherapy device. During use, the device sprays liquid carbon dioxide onto a patient to eradicate inflammation over a series of cryotherapy treatments.

The device is comprised of a body that is generally gun-like in shape. The body is preferably enclosed in an outer shell preferably made from a medical-grade plastic but may be made from any material. The outer shell may be comprised of at least one handle that increases grip.

The body is comprised of at least one quick-connect fitting that allows liquid CO2 to enter into the body. The flow of CO2 out of the body can be actuated via at least one trigger. More specifically, the trigger controls the operation of a valve, such as but not limited to a needle valve, that allows CO2 to exit the body when the valve is open. As the trigger is depressed, a flow path is created for the CO2 through the body to at least one nozzle.

The nozzle may be fixedly or removably attached to the body. The nozzle may have a plurality of spray patterns such as but not limited to fan, cone, etc. The spray patterns may be configured by turning the nozzle during use. In one embodiment, the device has a plurality of nozzles with various output levels meant for users who may need extra tissue penetration with the CO2 to get deep into the muscle to help eradicate inflammation in any of their body parts.

The body may also be comprised of at least one flow adjustment knob that increases and/or decreases the flow rate of CO2 through the body and allows a user to set a maximum flow rate. The flow rate is preferably between 1 psi and 1,000 psi. The body may also be comprised of at least one heating element that prevents the device from frosting over and creating dry ice and also ensures liquid CO2 expelled from the device comes out smoother.

The device may also be comprised of at least one thermistor that senses the temperature of the body and controls a heating element to prevent frosting of the exterior of the body due to excessive temperature drops. The device is also comprised of at least one temperature sensor that collects temperature data of objects in front of the device and sends the data back to a computer unit used to graphically indicate the dropping temperature of the patient's skin. Further, the device is comprised of at least one laser that projects in front of the device. The device may also be comprised of at least one connector that transmits power and communication between the device and a cable assembly while allowing quick separation for disassembly.

Accordingly, the high-pressure medical cryotherapy device of the present invention is particularly advantageous as it provides an improved cryotherapy device. More specifically, the device provides a high-pressure medical cryotherapy device that is portable. In addition, the device is well-built and durable. In this manner, the high-pressure medical cryotherapy device overcomes the limitations of existing cryotherapy devices known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a side cross-sectional view of one potential embodiment of a high-pressure medical cryotherapy device of the present invention in accordance with the disclosed architecture;

FIG. 2 illustrates a side cross-sectional view of one potential embodiment of a high-pressure medical cryotherapy device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for an improved cryotherapy device. There also exists a long-felt need in the art for a high-pressure medical cryotherapy device that is portable. In addition, there exists a long-felt need in the art for a high-pressure medical cryotherapy device that is well-built and durable.

The present invention, in one exemplary embodiment, is comprised of a high-pressure medical cryotherapy device that device sprays liquid carbon dioxide onto a patient to eradicate inflammation over a series of cryotherapy treatments. The device is comprised of a body that is generally gun-like in shape and is preferably enclosed in an outer shell preferably made from a medical-grade plastic but may be made from any material. The outer shell may be comprised of at least one handle that increases grip.

The body is comprised of at least one quick-connect fitting that allows liquid CO2 to enter into the body, wherein the flow of CO2 out of the body can be actuated via at least one trigger. More specifically, the trigger controls the operation of a valve, such as but not limited to a needle valve, that allows CO2 to exit the body when the valve is open. As the trigger is depressed, a flow path is created for the CO2 through the body to at least one nozzle.

The nozzle may be fixedly or removably attached to the body and may have a plurality of spray patterns such as but not limited to fan, cone, etc. The spray patterns may be configured by turning the nozzle during use. In one embodiment, the device has a plurality of nozzles with various output levels meant for users who may need extra tissue penetration with the CO2 to get deep into the muscle to help eradicate inflammation in any of their body parts.

The body may also be comprised of at least one flow adjustment knob that increases and/or decreases the flow rate of CO2 through the body and allows a user to set a maximum flow rate. The flow rate is preferably between 1 psi and 1,000 psi. The body may also be comprised of at least one heating element that prevents the device from frosting over and creating dry ice and also ensures liquid CO2 expelled from the device comes out smoother.

The device may also be comprised of at least one thermistor that senses the temperature of the body and controls a heating element to prevent frosting of the exterior of the body due to excessive temperature drops. The device is also comprised of at least one temperature sensor that collects temperature data of objects in front of the device and sends the data back to a computer unit used to graphically indicate the dropping temperature of the patient's skin. Further, the device is comprised of at least one laser that projects in front of the device. The device may also be comprised of at least one connector that transmits power and communication between the device and a cable assembly while allowing quick separation for disassembly.

Accordingly, the high-pressure medical cryotherapy device of the present invention is particularly advantageous as it provides an improved cryotherapy device. More specifically, the device provides a high-pressure medical cryotherapy device that is portable. In addition, the device is well-built and durable. In this manner, the high-pressure medical cryotherapy device overcomes the limitations of existing cryotherapy devices known in the art.

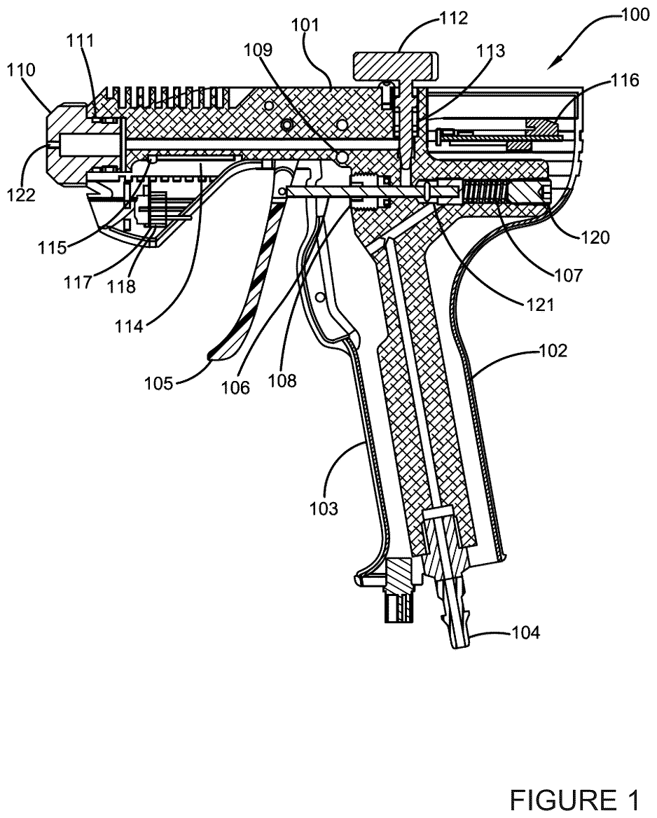

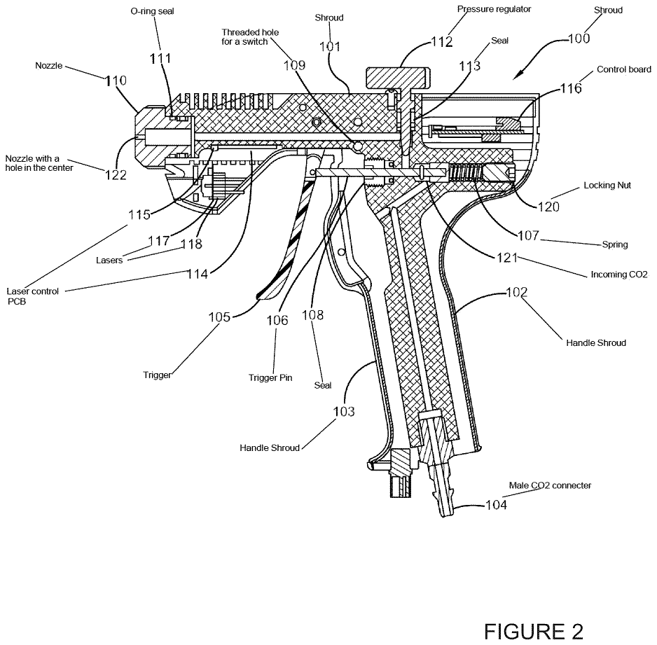

Referring initially to the drawings, FIG. 1 and FIG. 2 illustrates a side cross-sectional view of one potential embodiment of a high-pressure medical cryotherapy device 100 of the present invention in accordance with the disclosed architecture. During use, the device 100 sprays liquid carbon dioxide onto a patient to eradicate inflammation over a series of treatments. To do so, the liquid carbon dioxide creates vasoconstriction of the blood cells exposing the inflammation. Then, once cold gas hits the inflammation it reduces and suppresses the inflammation to achieve pain relief for the patient.

The device 100 is comprised of a body 101 that is generally gun-like in shape. The body 101 may be comprised of at least one rear seal 120 and at least one main seal 121. The main seal 121 seals the body 101 which is temporarily broken when the trigger 105 is depressed to allow CO2 to exit the nozzle 110 via at least one opening 122 of the nozzle 110. The rear seal 120 seals the rear side of the body 101. In the preferred embodiment, the body 101 is made from a solid piece of stainless steel body 101, although the body 101 may be made from any material. The body 101 is preferably enclosed in an outer shell 102. The outer shell 102 is preferably made from a medical-grade plastic but may be made from any material. The outer shell 102 protects the internal components of the device 100 and provides thermal insulation for the user's hand. The outer shell 102 may be comprised of at least one handle 103 that increases grip. The handle 103 may be made from a molded silicone but may be made from any material.

The body 101 is comprised of at least one quick-connect fitting 104. The fitting allows liquid CO2 to enter into the body 101. The quick-connect fitting 104 may be any type of quick-connect/quick-disconnect connector 119 known in the art. In one embodiment, the quick-connect fitting 104 is configured to connect to a bulk liquid CO2 supply tank rather than a disposable cartridge, thereby allowing extended treatment duration, increased treatment capacity, and reduced interruption during clinical procedures. The device 100 may therefore be configured for connection to CO2 tanks of various capacities through suitable pressure-rated fittings, hoses, regulators, and connector assemblies.

The flow of CO2 out of the body 101 can be actuated via at least one trigger 105 of any trigger 105 type/style. More specifically, the trigger 105 controls the operation of a valve 106, such as but not limited to a needle valve 106, which allows CO2 to exit the body 101 when the valve 106 is open. As the trigger 105 is depressed, a flow path is created for the CO2 through the body 101 to at least one nozzle. To assist in sealing the sale and returning the trigger 105 to an inoperative position, the trigger 105 may be comprised of at least-one trigger return spring 107. In addition, at least one trigger seal 108 provides a seal against the CO2 as the valve 106 protrudes towards the trigger 105. The trigger 105 may also be comprised of at least one trigger switch 109 located adjacent to and touching the trigger 105 that senses trigger 105 usage and sends trigger 105 status to a computer unit 116. In one embodiment, the trigger seal 108 is comprised of a Rulon® material. The Rulon trigger seal 108 may provide enhanced wear resistance, dimensional stability, low-friction operation, chemical resistance, and improved performance at extremely low temperatures associated with cryotherapy operation. The trigger seal 108 may therefore exhibit increased service life and improved sealing characteristics when compared to conventional polytetrafluoroethylene seal materials.

In one embodiment, the trigger 105 may further be comprised of an extended trigger body 124 having an increased length relative to conventional trigger configurations. The extended trigger body 124 may improve ergonomic engagement by users having different hand sizes while providing smoother trigger actuation and enhanced operational control.

In one embodiment, the trigger 105 is further associated with at least one safety lock assembly 125 comprised of a safety actuator 126, a locking member 127, and a safety spring 128. The safety spring 128 biases the locking member 127 into a normally engaged position that mechanically blocks movement of the trigger 105 and/or valve 106. Prior to activation of the trigger 105, a user may be required to intentionally actuate the safety actuator 126, thereby compressing the safety spring 128 and retracting the locking member 127 from the trigger pathway. While the safety actuator 126 remains engaged, the trigger 105 may actuate the valve 106 and permit cryotherapy discharge. Upon release of the safety actuator 126, the safety spring 128 automatically returns the locking member 127 to the engaged position to immediately prevent further actuation. The safety lock assembly 125 therefore provides a mechanical fail-safe system that reduces accidental discharge and requires coordinated user actions prior to operation.

The body 101 is comprised of at least one nozzle 110 that may be fixedly or removably attached to the body 101. The nozzle 110 may be any nozzle 110 type of any material. In the preferred embodiment, the nozzle 110 is press-fitted and locked into the body 101 without threads to prevent cross-threading and stripping. In one embodiment, the nozzle 110 is comprised of titanium or another high-strength material capable of withstanding repeated cryogenic temperature cycles and elevated operating pressures. The nozzle 110 may have a plurality of spray patterns such as but not limited to fan, cone, etc. The spray patterns may be configured by turning the nozzle 110 during use. In one embodiment, the device 100 has a plurality of nozzles with various output levels meant for users who may need extra tissue penetration with the CO2 to get deep into the muscle to help eradicate inflammation in any of their body 101 parts. The nozzle 110 is comprised of at least one seal (of any type) that seals the nozzle 110 to the body 101. The nozzle 110 may further cooperate with at least one nozzle locking assembly 129 comprised of a locking groove 130, at least one locking lug 131, at least one locking spring 132, and at least one alignment indicator 133. To install the nozzle 110, a user may align corresponding alignment indicators 133 positioned on the nozzle 110 and body 101, insert the nozzle 110 into a receiving cavity, and rotate the nozzle 110 in either a clockwise or counterclockwise direction. In one embodiment, approximately 180 degrees of rotational movement causes the locking lug 131 to engage the locking groove 130 under force from the locking spring 132. Once the alignment indicators 133 are aligned, the nozzle 110 is securely retained within the body 101. The nozzle locking assembly 129 reduces the likelihood of unintended nozzle separation or ejection during operation and provides a positive mechanical indication that the nozzle 110 is properly installed.

The body 101 may also be comprised of at least one flow adjustment knob 112. The flow adjustment knob 112 increases and/or decreases the flow rate of CO2 through the body 101 and allows a user to set a maximum flow rate. The flow rate is preferably between 1 psi and 1,000 psi. The flow adjustment knob 112 is sealed via at least one flow control seal 113 of any type.

The body 101 may also be comprised of at least one heating element 114. The heating element 114 may be any type and configuration of heating element 114 (such as but not limited to a resistive heating element 114) that prevents the device 100 from frosting over and creating dry ice and also ensures liquid CO2 expelled from the device 100 comes out smoother. The device 100 may further be comprised of at least one upgraded circuit board assembly 134 positioned within the handheld portion of the device 100. The circuit board assembly 134 may be configured to accommodate increased electrical power demands associated with sensors, heating elements, communication systems, safety systems, monitoring systems, and other electronic components.

The device 100 may further be comprised of at least one inductor 135 electrically positioned between a battery assembly and the circuit board assembly 134. The inductor 135 may smooth, regulate, and stabilize electrical current supplied to the handheld device components. The inductor 135 may reduce electrical noise, minimize transient current fluctuations, improve operational consistency, and increase reliability of electronic subsystems.

The device 100 may also be comprised of at least one thermistor 115 that senses the temperature of the body 101 and controls a heating element 114 to prevent frosting of the exterior of the body 101 due to excessive temperature drop. The device 100 is also comprised of at least one temperature sensor 117 that collects temperature data of objects in front of the device 100 and sends the data back to a computer unit 116 used to graphically indicate the dropping temperature of the patient's skin. Further, the device 100 is comprised of at least one laser 118 that projects in front of the device 100. In one embodiment, the device 100 is comprised of two laser emitters 118 configured to project converging laser beams. The laser emitters 118 may be calibrated such that beam convergence occurs at approximately eight inches from the nozzle 110. The convergence point may provide a visual indication of a preferred treatment distance between the nozzle 110 and a treatment area of a patient. In another embodiment, the device 100 has two converging lasers 118 that serve as a visual distance reference for a user of the device 100. The device 100 may further be comprised of at least one proximity sensing system 136 that cooperates with the laser emitters 118 and/or computer unit 116 to monitor treatment distance and provide user feedback when the device 100 is positioned outside a desired operating range.

The device 100 may further be comprised of at least one assembly board 137 mounted on or adjacent to a CO2 tank assembly. The assembly board 137 may be electrically connected to the computer unit 116 and may function as a centralized monitoring and control platform. The assembly board 137 may continuously monitor treatment duration, treatment segment timing, distance measurements obtained from the proximity sensing system 136, surface temperature measurements obtained from the temperature sensor 117, system operating status, fault conditions, and other treatment parameters. Data collected by the assembly board 137 may be displayed, recorded, transmitted, analyzed, or utilized to assist users in maintaining consistent treatment procedures.

The device 100 may further be comprised of at least one power switch 138 configured to selectively activate or deactivate device operation. The power switch 138 may be positioned on the body 101, outer shell 102, or another accessible location to permit rapid shutdown during emergency situations or routine operation.

The device 100 may also be comprised of at least one connector 119 that transmits power and communication between the device 100 and a cable assembly, while allowing quick separation for disassembly. In one embodiment, the connector 119 is enlarged relative to conventional connectors and is positioned at an angle of approximately forty-five degrees relative to a longitudinal axis of the body 101. Such positioning may improve user access, reduce cable strain, facilitate connection and disconnection, and improve ergonomic operation of the device 100.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “high-pressure medical cryotherapy device” and “device” are interchangeable and refer to the high-pressure medical cryotherapy device 100 of the present invention.

Notwithstanding the forgoing, the high-pressure medical cryotherapy device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the high-pressure medical cryotherapy device 100 as shown in the FIGS. are for illustrative purposes only, and that many other sizes and shapes of the high-pressure medical cryotherapy device 100 are well within the scope of the present disclosure. Although the dimensions of the high-pressure medical cryotherapy device 100 are important design parameters for user convenience, the high-pressure medical cryotherapy device 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A high-pressure medical cryotherapy device comprising:
a body;
a trigger;
a valve;
a nozzle;
a nozzle locking safety mechanism;
a flow adjustment knob;
a heating element; and
a quick-connector fitting.

2. The high-pressure medical cryotherapy device of claim 1, wherein the body is comprised of a gun-like shape.

3. The high-pressure medical cryotherapy device of claim 1 further comprised of a main seal.

4. The high-pressure medical cryotherapy device of claim 1, wherein the nozzle is comprised of an opening.

5. The high-pressure medical cryotherapy device of claim 1, wherein the body is comprised of a handle.

6. The high-pressure medical cryotherapy device of claim 1, wherein the valve is comprised of a needle valve.

7. The high-pressure medical cryotherapy device of claim 1 further comprised of a trigger switch.

8. A high-pressure medical cryotherapy device comprising:
a body comprised of an outer shell;
a trigger;
a valve;
a nozzle;
a trigger safety lock assembly;
a thermistor;
a computer unit;
a laser;
a flow adjustment knob;
a heating element; and
a quick-connector fitting.

9. The high-pressure medical cryotherapy device of claim 8 further comprised of a rear seal.

10. The high-pressure medical cryotherapy device of claim 8, wherein the nozzle is comprised of an adjustable spray pattern.

11. The high-pressure medical cryotherapy device of claim 8, wherein the nozzle is comprised of a nozzle seal.

12. The high-pressure medical cryotherapy device of claim 8, wherein the flow adjustment knob is comprised of a flow control seal.

13. The high-pressure medical cryotherapy device of claim 8, wherein the heating element is comprised of a resistive heating element.

14. The high-pressure medical cryotherapy device of claim 8 further comprised of a temperature sensor.

15. A high-pressure medical cryotherapy device comprising:
a body comprised of an outer shell;
a trigger;
a valve;
a nozzle;
an electrical current stabilization inductor;
a proximity sensing system;
a thermistor;
a connector;
a computer unit;
a first laser;
a flow adjustment knob;
a heating element; and
a quick-connector fitting.

16. The high-pressure medical cryotherapy device of claim 15 further comprised of a second laser.

17. The high-pressure medical cryotherapy device of claim 16, wherein the second laser converges with the first laser.

18. The high-pressure medical cryotherapy device of claim 15 further comprised of a temperature sensor.

19. The high-pressure medical cryotherapy device of claim 15, wherein the nozzle is removably attached to the body.

20. The high-pressure medical cryotherapy device of claim 15, wherein the nozzle has a cone spray pattern or a fan spray pattern.
